(19) United States
(12) Patent Application Publication
(10) Pub. No.: US 2022/0160964 A1
(43) Pub. Date: May 26, 2022

(54) AUTO-INJECTOR WITH LOCKING CLIP

(71) Applicant: Becton Dickinson France, Le Pont-de-Claix (FR)

(72) Inventor: Nicolas Besson, Treffort (FR)

(21) Appl. No.: 17/425,980

(22) PCT Filed: Feb. 26, 2020
(86) PCT No.: PCT/EP2020/055005

§ 371 Date: Jul. 27, 2021

(30) Foreign Application Priority Data
Feb. 26, 2019 (EP) 19305223.0

(51) Int. Cl. A61M 5/20; A61M 5/32; A61M 5/315; A61M 5/31

(52) U.S. Cl. CPC A61M 5/20; A61M 5/3202; A61M 5/31501; A61M 2005/2073; A61M 2205/12; A61M 2205/581; A61M 5/3129

(57) ABSTRACT

A drug delivery device includes a first subassembly having a cap, a needle cover, a syringe holder configured to receive a syringe assembly, a cassette body, and a lower housing shell, with the cap receiving at least a portion of the needle cover, a second subassembly, including a drive assembly configured to move a stopper of a syringe assembly, and a motor body, with the motor body receiving at least a portion of the drive assembly and the cassette body of the first subassembly configured to receive at least a portion of the motor body, and a locking clip configured to secure the first subassembly to the second subassembly upon assembly of the first subassembly to the second subassembly.

### CROSS-REFERENCE TO RELATED APPLICATIONS

This application is the United States national phase of International Application No. PCT/EP2020/055005 filed Feb. 26, 2020, and claims priority to Europe patent Application No. 19305223.0 filed Feb. 26, 2019, the entire disclosures of which are hereby incorporated by reference in their entirety.

### BACKGROUND OF THE INVENTION

### Field of the Disclosure

The present disclosure relates generally to a drug delivery device and, more specifically, to an auto-injector.

### Description of the Related Art

Various types of automatic injection devices have been developed to allow drug solutions and other liquid therapeutic preparations to be administered by untrained personnel or to be self-injected. Generally, these devices include a reservoir that is pre-filled with the liquid therapeutic preparation, and some type of automatic needle-injection mechanism that can be triggered by the user. Many of these devices, such as auto-injectors, are designed so that the reservoir, such as a pre-filled syringe, is assembled into the device during assembly of the device. In addition to automatically deploying the needle-injection mechanism, many drug delivery devices also automatically shield the needle after use of the device to prevent any unintended contact with the needle.

### SUMMARY OF THE INVENTION

In one aspect, a drug delivery device includes a first subassembly including a cap, a needle cover, a syringe holder configured to receive a syringe assembly, a cassette body, and a lower housing shell, with the cap receiving at least a portion of the needle cover, a second subassembly including a drive assembly configured to move a stopper of a syringe assembly, and a motor body, with the motor body receiving at least a portion of the drive assembly and the cassette body of the first subassembly configured to receive at least a portion of the motor body, and a locking clip configured to secure the first subassembly to the second subassembly upon assembly of the first subassembly to the second subassembly.

The drug delivery device may further include a syringe assembly including a barrel, a stopper, a cannula, and a rigid needle shield receiving at least a portion of the cannula.

The locking clip may include a body having a projection configured to engage a clip surface defined by the motor body of the second subassembly. The cassette body may define a clip opening configured to receive a portion of the locking clip. The locking clip may be C-shaped. The clip surface of the motor body may be defined by a recessed portion of the motor body, with the clip opening of the cassette body configured to be aligned with the clip surface of the motor body when the first subassembly is assembled with the second subassembly, with a portion of the locking clip configured to extend through the clip opening and the recessed portion of the motor body, with the projection of the locking clip engaging the clip surface of the motor body. The cassette body may define first and second grooves and the motor body defines first and second ribs, with the first groove of the cassette body configured to receive the first rib of the motor body, and the second groove of the cassette body configured to receive the second rib of the motor body.

The locking clip may be formed integrally with the cassette body. The motor body may define a clip surface configured to engage the locking clip of the cassette body. The device may further include an upper housing shell configured to be positioned adjacent to the lower housing shell, with the upper housing shell configured to prevent disengagement of the locking clip from the clip surface when the upper housing shell is engaged with the lower housing shell. The motor body may include a first extension defining a first channel and the cassette body may define a second extension defining a second channel, with the first channel of the motor body configured to receive the second extension of the cassette body. The second channel of the cassette body may be configured to receive the first extension of the motor body. The locking clip may include a first locking clip positioned on a first side of the cassette body and a second locking clip positioned on a second side of the cassette body.

In a further aspect, a method of assembling a drug delivery device includes: moving a first subassembly into engagement with a second subassembly, with the first subassembly including a cap, a needle cover, a syringe holder configured to receive a syringe assembly, a cassette body, and a lower housing shell, and the second subassembly including a drive assembly configured to move a stopper of a syringe assembly, and a motor body; securing the first subassembly to the second subassembly with a locking clip; and moving an upper housing shell into engagement with the second subassembly.

The method may further include positioning a syringe assembly in the syringe holder of the first subassembly, with the syringe assembly including a barrel, a stopper, a cannula, and a rigid needle shield receiving at least a portion of the cannula.

The method may further include: inserting a portion of the locking clip through the clip opening of the cassette body and engaging the clip surface of the motor body to secure the cassette body to the motor body.

The method may further include: positioning the motor body within the cassette body until the locking clip of the cassette body engages the clip surface of the motor body thereby securing the first subassembly to the second subassembly. Moving the upper housing shell into engagement with the lower housing shell may prevent disengagement of the locking clip from the clip surface.

The device may include one or several of the following features, taken individually or according to all technical possible combinations:
a drug delivery device may comprise: a first subassembly comprising a cap, a needle cover, a syringe holder configured to receive a syringe assembly, a cassette body, and a lower housing shell, the cap receiving at least a portion of the needle cover; a second subassembly comprising a drive assembly configured to move a stopper of a syringe assembly, and a motor body, the motor body receiving at least a portion of the drive assembly, the cassette body of the first subassembly is configured to receive at least a portion of the motor body; and a locking clip configured to secure the first subassembly to the second subassembly upon assembly of the first subassembly to the second subassembly;
the drug delivery device may comprise a syringe assembly comprising a barrel, a stopper, a cannula, and a rigid needle shield receiving at least a portion of the cannula;
the locking clip may comprise a body having a projection configured to engage a clip surface defined by the motor body of the second subassembly;
the cassette body may define a clip opening configured to receive a portion of the locking clip;
the locking clip may be C-shaped;
the clip surface may be defined by a recessed portion of the motor body, the clip opening of the cassette body is configured to be aligned with the clip surface of the motor body when the first subassembly is assembled with the second subassembly, a portion of the locking clip configured to extend through the clip opening of the cassette body and the recessed portion of the motor body with the projection of the locking clip engaging the clip surface of the motor body;
the cassette body may define first and second grooves and the motor body defines first and second ribs, the first groove of the cassette body configured to receive the first rib of the motor body, and the second groove of the cassette body configured to receive the second rib of the motor body;
the locking clip is formed integrally with the cassette body;
the locking clip may include a first locking clip positioned on a first side of the cassette body and a second locking clip positioned on a second side of the cassette body;
the motor body may define a clip surface configured to engage the locking clip(s) of the cassette body;
an upper housing shell may be configured to be positioned adjacent to the lower housing shell;
the motor body may include a first extension defining a first channel and the cassette body defines a second extension defining a second channel, the first channel of the motor body configured to receive the second extension of the cassette body;
the second channel of the cassette body may be configured to receive the first extension of the motor body.

Another aspect of the invention concerns a method of assembling a drug delivery device comprising: moving a first subassembly into engagement with a second subassembly, the first subassembly comprising a cap, a needle cover, a syringe holder configured to receive a syringe assembly, a cassette body, and a lower housing shell, the second subassembly comprising a drive assembly configured to move a stopper of a syringe assembly, and a motor body; securing the first subassembly to the second subassembly with a locking clip; and moving an upper housing shell into engagement with the second subassembly.

The method may also comprise one or several of the following features, taken individually or according to all technical possible combinations:
the method may comprise positioning a syringe assembly in the syringe holder of the first subassembly, the syringe assembly comprising a barrel, a stopper, a cannula, and a rigid needle shield receiving at least a portion of the cannula;
the locking clip may comprise a body having a projection and the motor body defines a clip surface, and wherein the cassette body defines a clip opening configured to receive a portion of the locking clip, the method further comprising: inserting a portion of the locking clip through the clip opening of the cassette body and engaging the clip surface of the motor body to secure the cassette body to the motor body;
the locking clip may be formed integrally with the cassette body, and wherein the motor body defines a clip surface, the method further comprising: positioning the motor body within the cassette body until the locking clip of the cassette body engages the clip surface of the motor body thereby securing the first subassembly to the second subassembly.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1A is a perspective view of a drug delivery device according to one aspect of the present application, showing a storage position of the device.

FIG. 1B is a perspective view of the drug delivery device of FIG. 1, showing a pre-use position of the device.

FIG. 2A is a cross-sectional view of the drug delivery device of FIG. 1, showing a storage position of the device.

FIG. 2B is a cross-sectional view of the drug delivery device of FIG. 1, showing a pre-use position of the device.

FIG. 3 is a perspective view of the drug delivery device of FIG. 1, showing an actuation position of the device.

FIG. 4 is a cross-sectional view of the drug delivery device of FIG. 1, showing an actuation position of the device.

FIG. 5 is a perspective view of the drug delivery device of FIG. 1, showing an injection position of the device.

FIG. 6 is a cross-sectional view of the drug delivery device of FIG. 1, showing an injection position of the device.

FIG. 7 is a perspective view of the drug delivery device of FIG. 1, showing a post-use position of the device.

FIG. 8 is a cross-sectional view of the drug delivery device of FIG. 1, showing a post-use position of the device.

FIG. 9 is a perspective view of the drug delivery device of FIG. 1, showing a locking clip.

FIG. 10 is an exploded perspective view of the drug delivery device of FIG. 1, showing a locking clip.

FIG. 11A is a partial cross-sectional view of the drug delivery device of FIG. 1, showing a lock arm of a cassette body.

FIG. 11B is an enlarged cross-sectional view of the area indicated in FIG. 11A.

FIG. 12 is a cross-sectional view of the drug delivery device of FIG. 1, showing a post-use position of the device prior to full delivery of medicament.

FIG. 13 is a bottom perspective view of a cassette body of the drug delivery device of FIG. 1.

FIG. 14 is a top perspective view of a locking clip of the drug delivery device of FIG. 1.

FIG. 15 is a perspective view of a motor body and locking clip of the drug delivery device of FIG. 1.

FIG. 16 is a bottom perspective view of a cassette body and motor body according to a further aspect of the present application.

FIG. 17 is a perspective view of the cassette body of FIG. 16.

FIG. 18 is a partial side view of a drug delivery device according to a further aspect of the present application, showing the cassette body and motor body of FIG. 16.

FIG. 19 is a partial perspective view of the cassette body and motor body of FIG. 16.

FIG. 20 is a partial perspective view of the drug delivery device of FIG. 18.

FIG. 21 is a perspective view of a drug delivery device according to a further aspect of the present application, showing a storage position of the device.

FIG. 22 is a cross-sectional view taken along line 22-22 shown in FIG. 21.

FIG. 23 is a top perspective view of a motor body of the drug delivery device of FIG. 21.

FIG. 24 is a bottom perspective view of the motor body of FIG. 23.

FIG. 25 is a front perspective view of a plunger body of the drug delivery device of FIG. 21.

FIG. 26 is a rear perspective view of the plunger body of FIG. 25.

FIG. 27 is a front perspective view of a plunger rod portion of the drug delivery device of FIG. 21.

FIG. 28 is a rear perspective view of the plunger rod portion of FIG. 27.

FIG. 29 is a top perspective view of a lever actuation member of the drug delivery device of FIG. 21.

FIG. 30 is a bottom perspective view of the lever actuation member of the drug delivery device of FIG. 29.

FIG. 31 is a front perspective view of a syringe holder of the drug delivery device of FIG. 21.

FIG. 32 is a rear perspective view of the syringe holder of the drug delivery device of FIG. 31.

FIG. 33 is a front perspective view of a needle cover of the drug delivery device of FIG. 21.

FIG. 34 is a rear perspective view of the needle cover of the drug delivery device of FIG. 33.

FIG. 35 is a top perspective view of a cassette body of the drug delivery device of FIG. 21.

FIG. 36 is a bottom perspective of the cassette body of the drug delivery device of FIG. 35.

FIG. 37 is a top perspective view of a cap of the drug delivery device of FIG. 21.

FIG. 38 is a cross-sectional view taken along line 38-38 in FIG. 37.

FIG. 39 is a perspective view of a retainer of the drug delivery device of FIG. 21.

FIG. 40 is a cross-sectional view of an upper housing shell of the drug delivery device of FIG. 21.

FIG. 41 is perspective view of a lower housing shell of the drug delivery device of FIG. 21.

FIG. 42 is a cross-sectional view taken along line 42-42 in FIG. 41.

FIG. 43 is a cross-sectional view of the drug delivery device of FIG. 21, showing an injection position of the device.

FIG. 44 is a partial cross-sectional view of the drug delivery system of FIG. 21.

FIG. 45 is a cross-sectional view of the drug delivery system of FIG. 21.

Corresponding reference characters indicate corresponding parts throughout the several views. The exemplifications set out herein illustrate exemplary aspects of the disclosure, and such exemplifications are not to be construed as limiting the scope of the disclosure in any manner.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

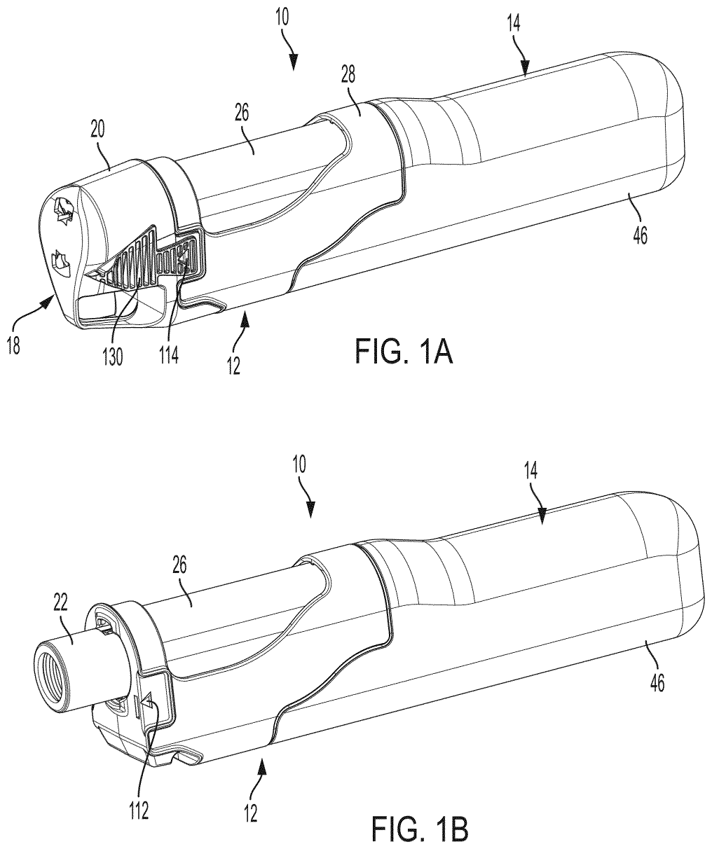

Referring to FIGS. 1A-10 a drug delivery device 10 according to one aspect of the present invention includes a first subassembly 12, a second subassembly 14, and a syringe assembly 16. The first subassembly 12 includes a cap 18 having an outer portion 20, a needle cover 22, a syringe holder 24, a cassette body 26, and a lower housing shell 28. The second subassembly 14 includes a drive assembly 40, a motor body 42, a lever actuation member 44, and an upper housing shell 46. The syringe assembly 16 is received by the syringe holder 24 and includes a barrel 52, a stopper 54, a cannula 56, and a rigid needle shield (RNS) 58. The lower housing shell 28, the cassette body 26, and the upper housing shell 46 generally form a housing for receiving the various components of the device 10, although other suitable housing arrangements may be utilized. As discussed in more detail below, the first subassembly 12 and the second subassembly 14 are secured to each other during assembly by a locking clip 64, although other suitable arrangements may be utilized. The drug delivery device 10 may be an auto-injector, although the features described herein may be incorporated into other suitable drug delivery devices.

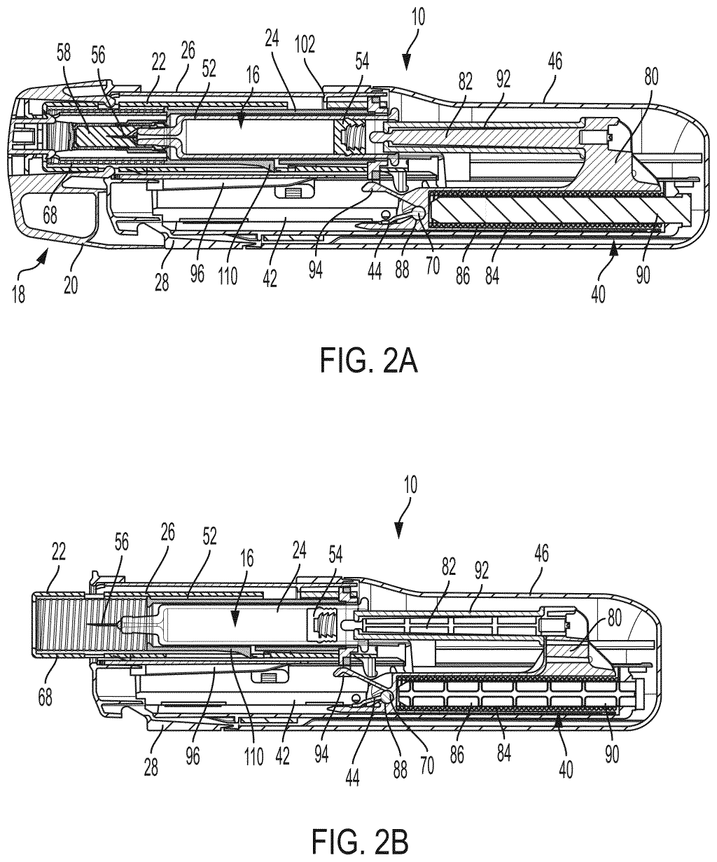

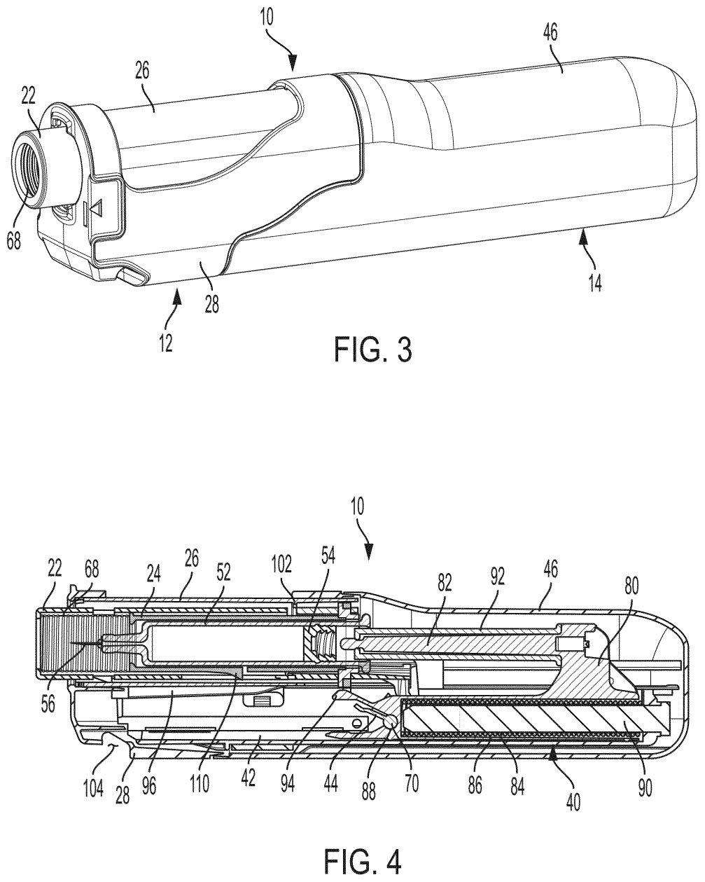

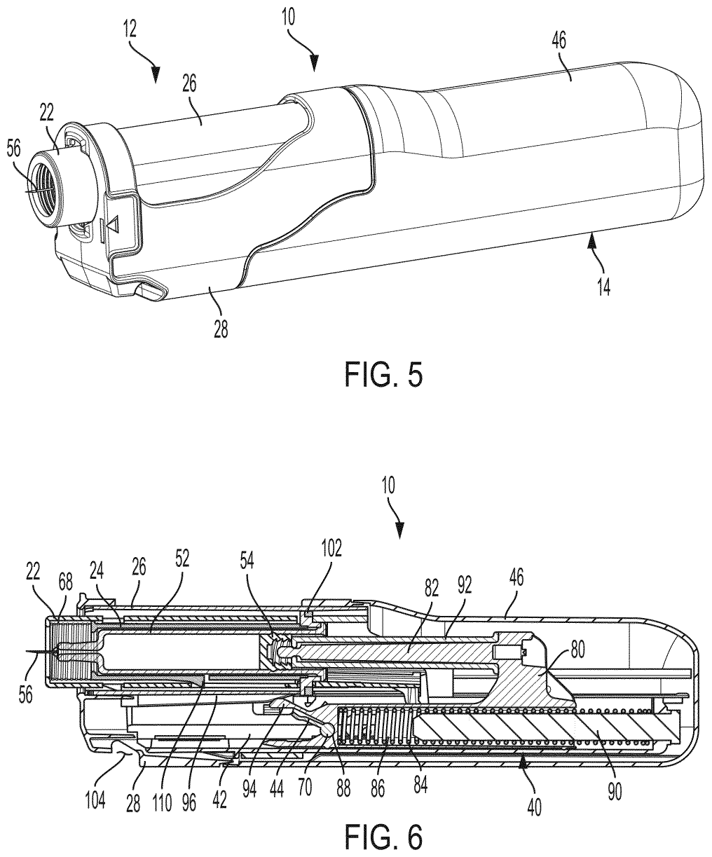

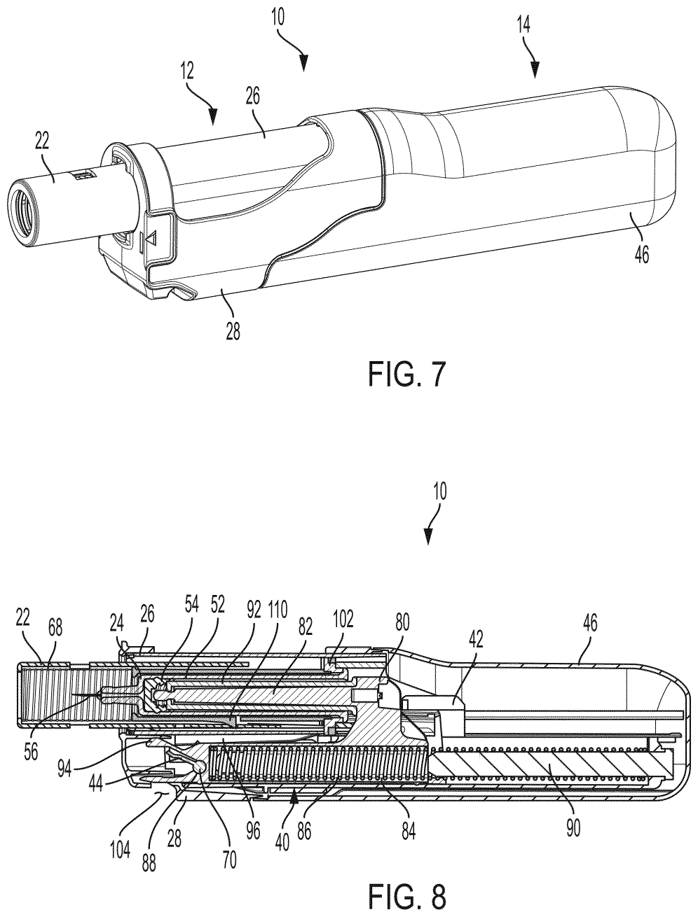

The drug delivery device 10 is configured to automatically deliver a dose of medicament from the syringe assembly 16 to a patient upon actuation of the device 10. More specifically, upon actuation of the drug delivery device 10, the drive assembly 40 is configured to engage the stopper 54 of the syringe assembly 16, displace the syringe assembly 16 such that the cannula 56 pierces the skin of the patient, and displace the stopper 54 within the barrel 52 of the syringe assembly 16 to deliver the medicament within the barrel 52. The drug delivery device 10 includes a storage position (FIGS. 1A and 2A), a pre-use position (FIGS. 1B and 2B), an actuation position (FIGS. 3 and 4), an injection position (FIGS. 5 and 6), and a post-use position (FIGS. 7 and 8). As discussed in more detail below, the needle cover 22 is configured to shield the cannula 56 of the syringe assembly 16 from the patient when the device 10 is in the pre-use and the post-use positions. In particular, the needle cover 22 is movable between a pre-use position, an actuation position, and a post-use position, with a spring 68 biasing the needle cover 22 towards the pre-use position and the post-use position. The spring 68 is positioned between the needle cover 22 and the syringe holder 24, although other suitable arrangements may be utilized. The lever actuation member 44 is movable between a locked position where movement of the drive assembly 40 is prevented and a released position where movement of the drive assembly 40 is allowed. More specifically, the lever actuation member 44 is rotatable about a rotation axis 70 between the locked position and the released position. When the lever actuation member 44 is in the locked position, the lever actuation member 44 is engaged with the motor body 42 and the drive assembly 40 to prevent movement of the drive assembly 40. When the lever actuation member 44 is in the released position, the lever actuation member 44 is disengaged from the motor body 42 thereby allowing movement of the drive assembly 40 toward the syringe assembly 16. The rotation axis 70 of the lever actuation member 44 extends perpendicular to a longitudinal axis of the device 10, although other suitable arrangements may be utilized.

Referring again to FIGS. 1-10, the drive assembly 40 includes a plunger body 80 having a plunger rod portion 82 and a drive member 84. The drive member 84 is a compression spring received within a drive opening 86 defined by the plunger body 80, although other suitable drive members may be utilized, including, but not limited to, compressed gas, an electric motor, hydraulic pressure, other types of springs, etc. The drive member 84 engages the plunger body 80 and the motor body 42 and biases the plunger body 80 in a direction extending from the second subassembly 14 toward the first subassembly 12. The plunger body 80 defines a lever opening 88 that receives the lever actuation member 44 and defines the rotation axis 70 of the lever actuation member 44. The lever actuation member 44 prevents movement of the plunger body 80 when the lever actuation member 44 is in the locked position through engagement of the lever actuation member 44 with the motor body 42. Upon rotation of the lever actuation member 44 from the locked position to the released position, the lever actuation member 44 is disengaged from the motor body 42 thereby allowing the drive member 84 to move the plunger body 80 and the plunger rod portion 82 toward the first subassembly 12. The plunger rod portion 82 and the drive member 84 are spaced from and parallel to each other and extend in a longitudinal direction of the device 10.

The drive assembly 40 further includes a spring guide member 90 secured to the upper housing shell 46 and received within the drive opening 86 of the plunger body 80. The drive member 84 is received by the spring guide member 90 such that the drive member 84 is positioned between the plunger body 80 and the spring guide member 90. The drive assembly 40 also includes a plunger rod cover 92 that receives the plunger rod portion 82 of the plunger body 80. The plunger rod cover 92 is configured to guide insertion of the plunger rod portion 82 into the barrel 52 of the syringe assembly 16 and engage the stopper 54 of the syringe assembly 16 to dispense the medicament from the barrel 52 of the syringe assembly 16. The plunger rod cover 92 and the plunger rod portion 82 may be formed integrally or formed as separate components.

The plunger body 80 of the drive assembly 40 also includes an audio indicator member 94 configured to provide an audible indication to a user when the device 10 transitions to the post-use position. As discussed in more detail below, the audio indicator member 94 is configured to engage one or more ribs 96 of the cassette body 26 when the device 10 is in the injection position thereby deflecting the audio indicator member 94. When the drug delivery device 10 transitions from the injection position to the post-use position, the audio indicator member 94 disengages from the rib(s) 96 of the cassette body 26 and contacts the lower housing shell 28 to provide an audible click, although the audio indicator member 94 could also contact other suitable portions of the device 10 to provide the audible indicator.

Referring to FIGS. 1A-2B, in the storage position, the cap 18 is secured to the lower housing shell 28 and engaged with the needle cover 22. Movement of the needle cover 22 from the pre-use position to the actuation position causes engagement between the needle cover 22 and the lever actuation member 44 thereby actuating the drive assembly 40. After removal of the cap 18 by grasping the outer portion 20, the needle cover 22 may be moved from the pre-use position to the actuation position by pressing the needle cover 22 against a skin surface of a patient and axially pressing the device 10 against the skin surface. As detailed below, the engagement between the cap 18 and the needle cover 22 prevents the needle cover 22 from moving into engagement with the lever actuation member 44. Accordingly, removal of the cap 18 from the device 10 allows for the actuation of the device 10. Removal of the cap 18 from the device 10, as shown in FIGS. 1B and 2B, also removes the RNS 58 from the syringe barrel 52 thereby exposing the cannula 56, which is still received within the needle cover 22 in the pre-use position of the device 10. The cap 18 may include one or more components received within the outer portion 20 to facilitate the removal of the RNS 58.

Referring to FIGS. 3 and 4, in the actuation position, the cap 18 is removed and the needle cover 22 is positioned in the actuation position by engaging a skin surface of a patient, which moves the needle cover 22 further within the device 10 toward the second subassembly 14. When the needle cover 22 has moved a sufficient distance within the device 10, a portion of the needle cover 22 engages the lever actuation member 44, which rotates the lever actuation member 44 about the rotation axis 70 from the locked position to the released position.

Referring to FIGS. 5 and 6, in the injection position, the lever actuation member 44 is in the released position, which allows the plunger body 80 of the drive assembly 40 to move toward the first subassembly 12 such that the plunger body 80 or the plunger rod cover 92 engages the stopper of the syringe assembly 16. Initial engagement of the drive assembly 40 with the syringe assembly 16 moves the syringe assembly 16 and the syringe holder 24 within the device 10 and relative to the cassette body 26 until the syringe holder 24 abuts a stop 102 defined by the cassette body 26. During this initial movement of the syringe assembly 16 and syringe holder 24 with the needle cover 22 pressed against a skin surface of a patient, the cannula 56 of the syringe assembly 16 extends beyond the needle cover 22 and pierces the skin surface of the patient. Further movement of the plunger body 80, which is driven by the drive member 84, moves the stopper 54 relative to the barrel 52 of the syringe assembly 16 to dispense medicament from the barrel 52 of the syringe assembly 16, through the cannula 56, and into the patient. The plunger body 80 will continue moving until the stopper 54 bottoms out on the barrel 52 of the syringe assembly 16. When the stopper 54 bottoms out, the audio indicator member 94 will disengage from the rib(s) 96 of the cassette body 26 and contact the lower housing shell 28 at approximately the same time to provide the audible indication to the patient that the dose of medicament has been delivered. In addition to the audible indication, the drug delivery device 10 provides one or more visual indicators to notify a patient of the status of the device 10. In particular, the cassette body 26 may be formed from transparent material to allow visual confirmation of movement of the stopper 54 and/or another visual indicator provided by the drive assembly 40, syringe holder 24, and/or syringe assembly 16. The lower housing shell 28 also defines an indicator opening 104, which provides visual indication that the plunger body 80 is in a final position and the dose of medicament has been delivered. The visual indicators may utilize contrasting colors, symbols, patterns, or any other suitable visual indicia to indicate the various statuses of the device.

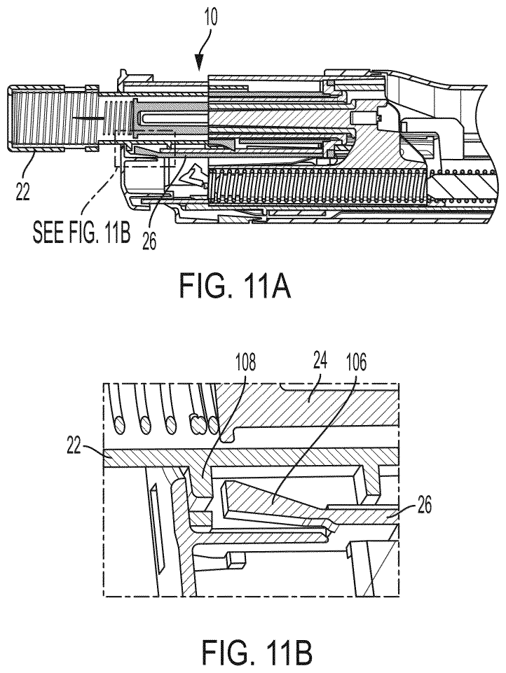

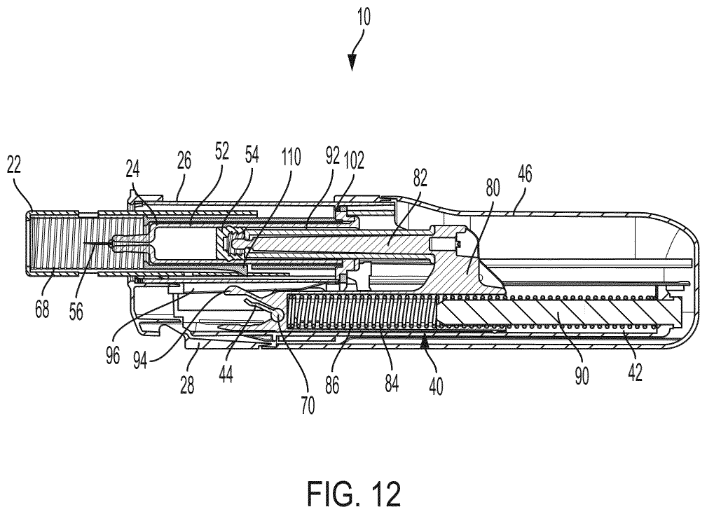

Referring to FIGS. 7, 8, 11A, 11B, and 12, in the post-use position, the needle cover 22 extends to the post-use position to shield the cannula 56 when the needle cover 22 is removed from a skin surface of a patient. As shown more clearly in FIG. 11B, the cassette body 26 includes a lock arm 106 and the needle cover 22 includes a lock protrusion 108, although other suitable configurations may be utilized. The lock arm 106 of the cassette body 26 engages the lock protrusion 108 of the needle cover 22 to prevent any further use of the device 10 and exposing of the cannula 56 of the syringe assembly 16. During the transition of the device 10 from the injection position to the post-use position, the lock arm 106 of the cassette body 26 deflects to allow the lock protrusion 108 of the needle cover 22 to pass by the cassette body 26 with the lock arm 106 returning to its original position to prevent movement of the needle cover 22 back toward the pre-use and actuation positions. In the pre-use position of the needle cover 22, a portion of the needle cover 22 engages a cover stop 110 of the syringe holder 24 to limit axial movement of the needle cover 22 in a direction extending from the second subassembly 14 toward the first subassembly 12. After use of the device 10, the syringe holder 24 is displaced within the cassette body 26 relative to the needle cover 22, which allows the needle cover 22 to extend to the post-use position when a patient removes the needle cover 22 from a skin surface. As shown in FIG. 12, the needle cover 22 will move to the post-use position when the needle cover 22 is removed from a skin surface of a patient regardless of a position of the stopper 54 within the barrel 52 of the syringe assembly 16. Accordingly, if a patient removes the needle cover 22 from a skin surface after only a portion of the dose of medicament has been delivered, the needle cover 22 will still move to the post-use position and will prevent further use of the device 10.

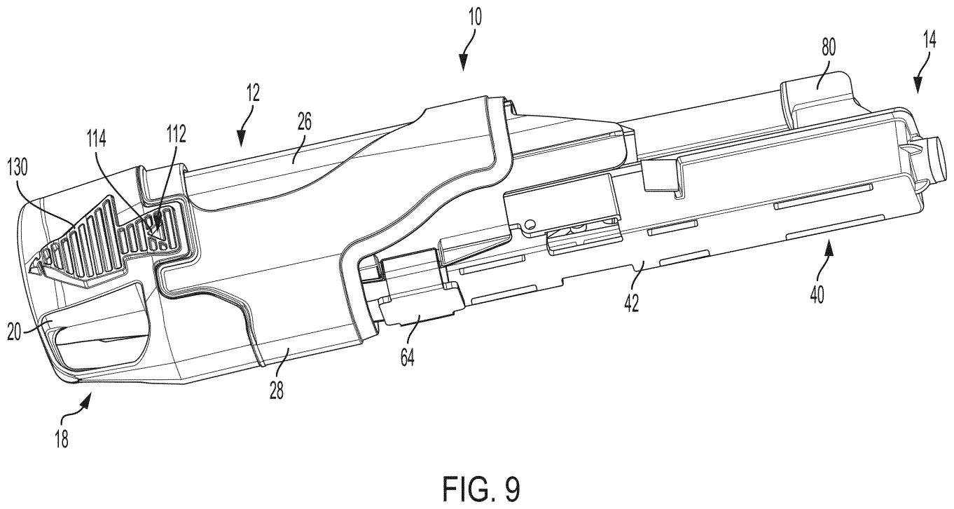

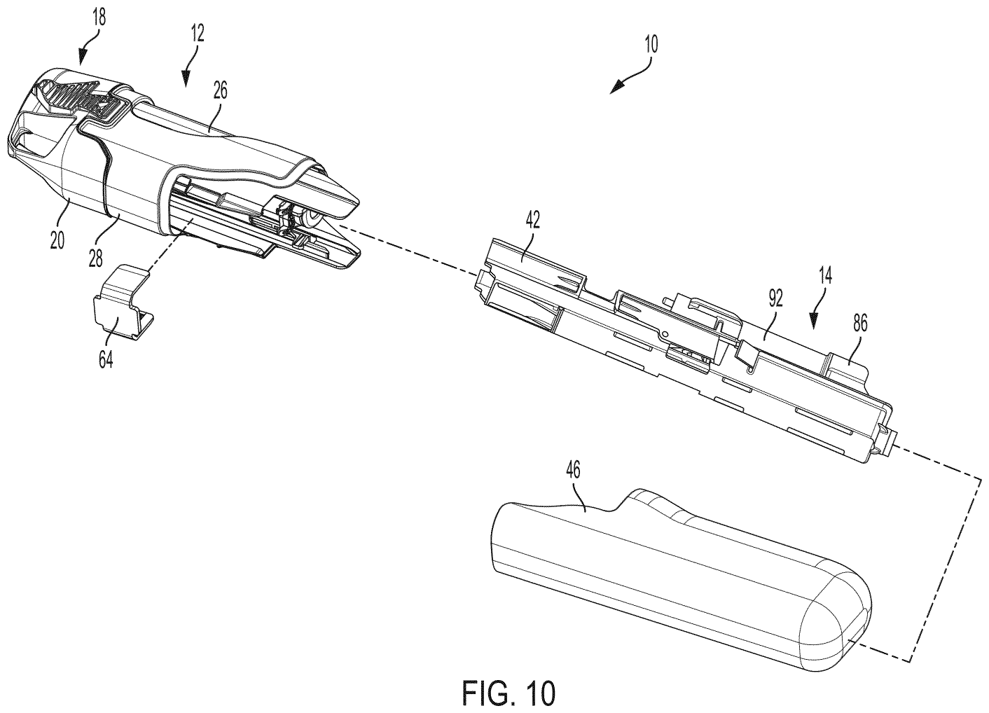

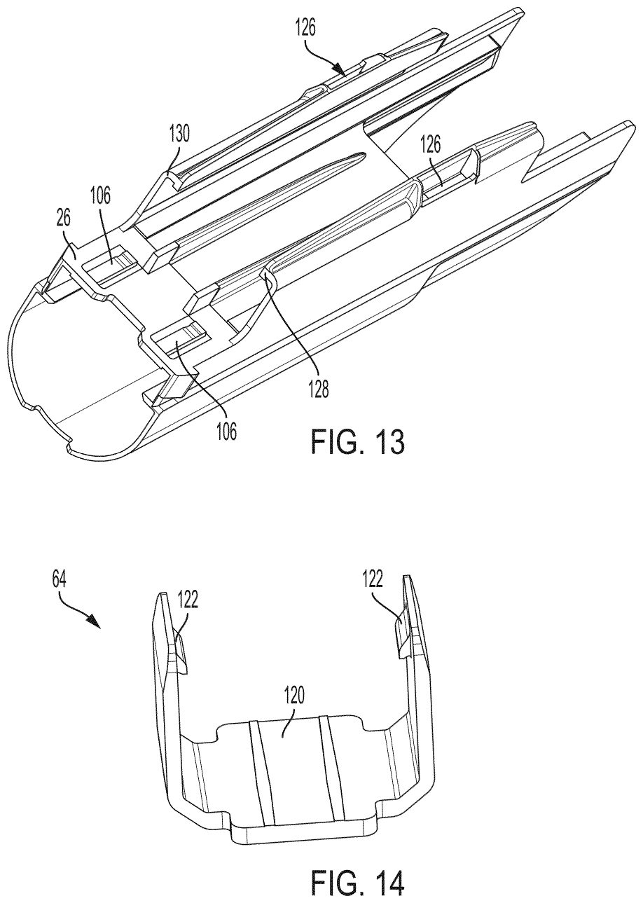

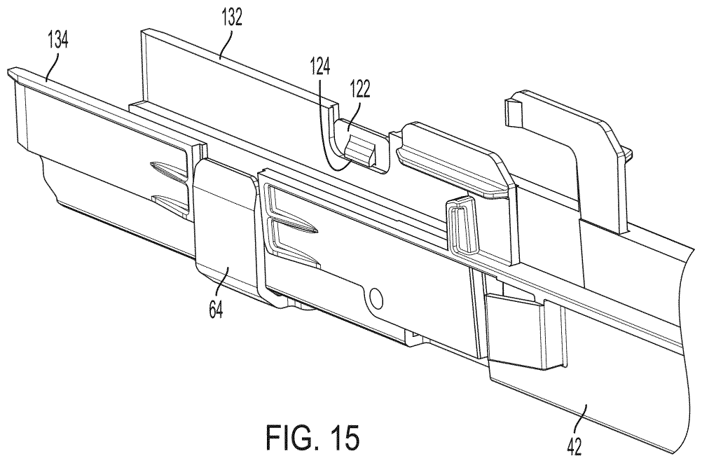

Referring to FIGS. 9, 10, and 13-15, the locking clip 64 is configured to secure the first subassembly 12 to the second subassembly 14 upon assembly of the first subassembly 12 to the second subassembly 14. As shown in FIG. 9, the cassette body 26 is configured to receive at least a portion of the motor body 42. Although not shown in FIG. 10, the syringe assembly 16 is positioned within the syringe holder 24 prior to assembly of the first subassembly 12 to the second subassembly 14. As shown in FIG. 14, the locking clip 64 includes a body 120 having at least one projection 122 configured to engage at least one clip surface 124 defined by the motor body 42 of the second subassembly 14. Referring to FIG. 15, the locking clip 64 includes two projections 122 and the motor body 42 includes two clip surfaces 124, although other suitable arrangements may be utilized. Further, as shown more clearly in FIG. 13, the cassette body 26 defines at least one clip opening 126 configured to receive a portion of the locking clip 64. As shown in FIG. 13, two clip openings 126 are provided, although other suitable arrangements may be utilized. The locking clip 64 is C-shaped, although other suitable shapes and configurations may be utilized. The clip openings 126 of the cassette body 26 are configured to be aligned with the clip surfaces 124 of the motor body 42 when the first subassembly 12 is assembled with the second subassembly 14. More specifically, in one aspect, the clip openings 126 are aligned with the clip surfaces 124 in a direction extending along a longitudinal axis of the device 10. A portion of the locking clip 64 is configured to extend through the clip openings 126 with the projections 122 of the locking clip 64 engaging the clip surfaces 124 of the motor body 42. The cassette body 26 defines first and second grooves 128, 130 and the motor body 42 defines first and second ribs 132, 134. The first groove 128 of the cassette body 26 is configured to receive the first rib 132 of the motor body 42 and the second groove 130 of the cassette body 26 is configured to receive the second rib 134 of the motor body 42.

Referring to FIGS. 9 and 10, a method of assembling the drug delivery device 10, according to one aspect of the present application, includes: moving the first subassembly 12 into engagement with the second subassembly 14; securing the first subassembly 12 to the second subassembly 14 with the locking clip 64; and moving the upper housing shell 46 into engagement with the lower housing shell 28. The first subassembly 12 is axially aligned with the second subassembly 14 and the motor body 42 is positioned within the cassette body 26 with the first and second subassemblies 12, 14 moved towards each other until the first and second grooves 128, 130 of the cassette body 26 receives the first and second ribs 132, 134 of the motor body 42. The first and second grooves 128, 130 of the cassette body 26 guide the insertion of the motor body 42 into the cassette body 26. The motor body 42 and cassette body 26 are moved toward each other until the clip openings 126 of the cassette body 26 are aligned with the clip surfaces 124 of the motor body 42, which may occur when the motor body 42 contacts the lower housing shell 28 of the first subassembly 12. Although not shown in FIG. 10, prior to assembly of the first and second subassemblies 12, 14, the syringe assembly 16 is positioned within the syringe holder 24. A portion of the body 120 of the locking clip 64 is inserted through the clip openings 126 of the cassette body 26 until the projections 122 engage the clip surfaces 124 of the motor body 42, which secures the cassette body 26 to the motor body 42 and the first subassembly 12 to the second subassembly 14.

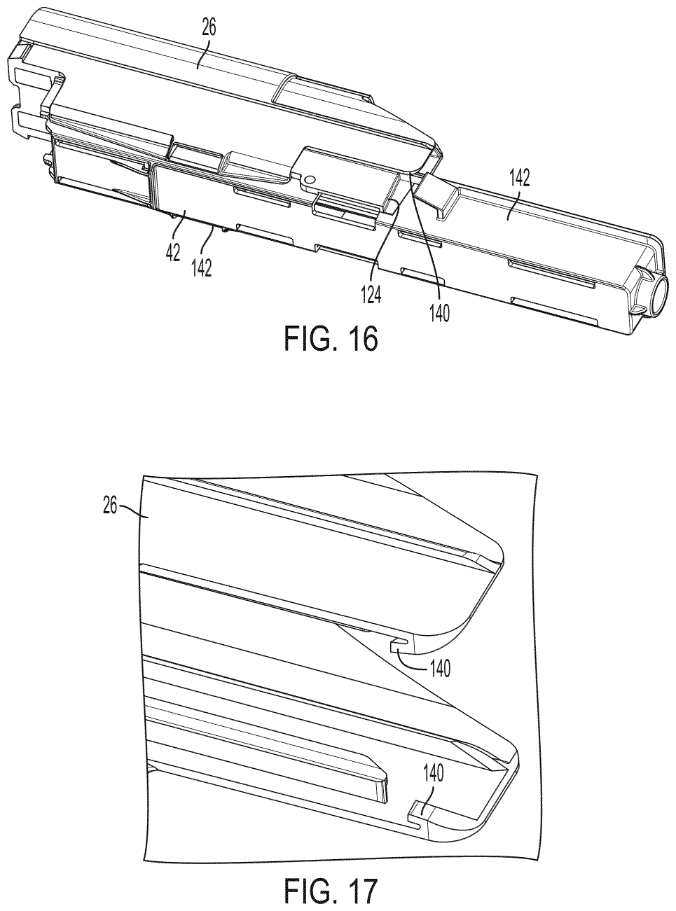

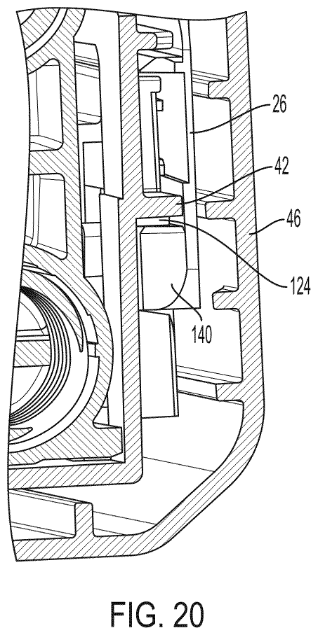

Referring to FIGS. 16-20, according to a further aspect of the present application, a locking clip 140 is formed integrally with the cassette body 26 for securing the first subassembly 12 to the second subassembly 14 rather than providing the separate locking clip 64 discussed above. More specifically, the cassette body 26 includes two locking clips 140 on each side of the cassette body 26, although one or more locking clips 140 may be provided. The locking clips 140 are configured to secure the first subassembly 12 to the second subassembly 14 upon assembly of the first subassembly 12 to the second subassembly 14 in a similar manner as the locking clip 64 of FIGS. 9 and 10. Side surfaces 142 of the motor body 42 defines the clip surfaces 124, which are configured to engage the locking clips 140 of the cassette body 26, although one or more clip surfaces 124 may be provided depending on the configuration of the locking clips 140. As the motor body 42 is received by the cassette body 26, the locking clips 140, shown more clearly in FIG. 17, are configured to deflect radially outward until the motor body 42 is fully inserted into the cassette body 26 and the locking clips 140 are aligned with the clip surfaces 124 thereby allowing the locking clips 140 to return to their original unbiased position. As shown in FIG. 20, the upper housing shell 46 is configured to prevent disengagement of the locking clips 140 from the clip surfaces 124 when the upper housing shell 46 is engaged with the lower housing shell 28. More specifically, when the upper housing shell 46 is positioned over the motor body 42 and engaged with the lower housing shell 28, the upper housing shell 46 prevents the locking clips 140 from deflecting radially outward, which prevents the locking clips 140 from being disengaged from the clip surfaces 124 of the motor body 42. When positioned over the cassette body 26 and the motor body 42, the upper housing shell 46 is positioned sufficiently close to the locking clips 140 to prevent such radial deflection and will engage the locking clips 140 upon movement of the locking clips 140.

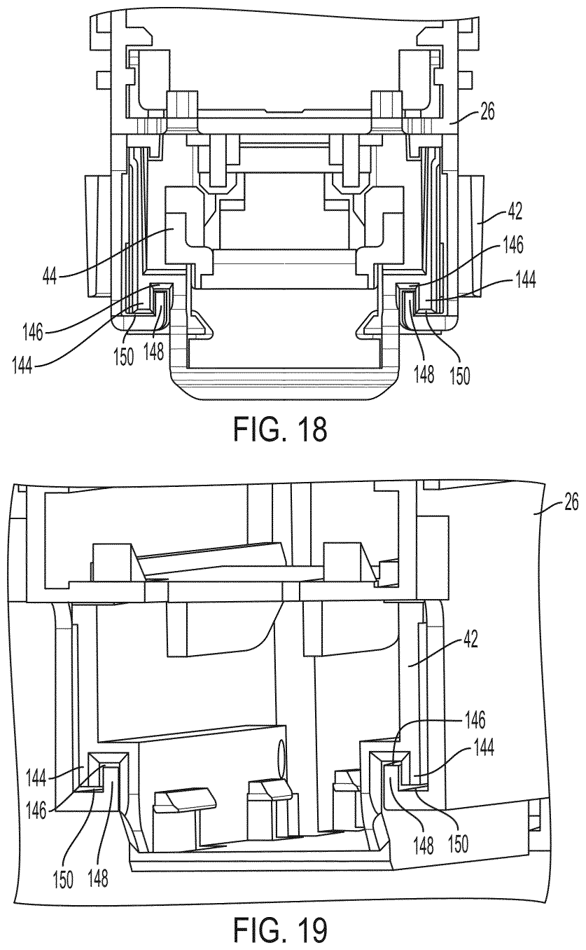

Referring to FIGS. 18 and 19, the motor body 42 includes a first extension 144 defining a first channel 146 and the cassette body 26 defines a second extension 148 defining a second channel 150. The first channel 146 of the motor body 42 is configured to receive the second extension 148 of the cassette body 26. The second channel 150 of the cassette body 26 is configured to receive the first extension 144 of the motor body 42. The motor body 42 includes two first extensions 144 and two first channels 146 and the cassette body 26 includes two second extensions 148 and two second channels 150, although one or more first and second extensions 144, 148 and one or more first and second channels 146, 150 may be provided.

Referring to FIG. 16, the first subassembly 12 is secured to the second subassembly 14 by positioning the motor body 42 within the cassette body 26 until the locking clips 140 of the cassette body 26 engage the clip surfaces 124 of the motor body 42 thereby securing the first subassembly 12 to the second subassembly 14. As noted above, moving the upper housing shell 46 into engagement with the lower housing shell 28 prevents disengagement of the locking clips 140 from the clip surfaces 124. The engagement between the first and second channels 146, 150 and the first and second extensions 144, 148, respectively, guides the insertion of the motor body 42 into the cassette body 26.

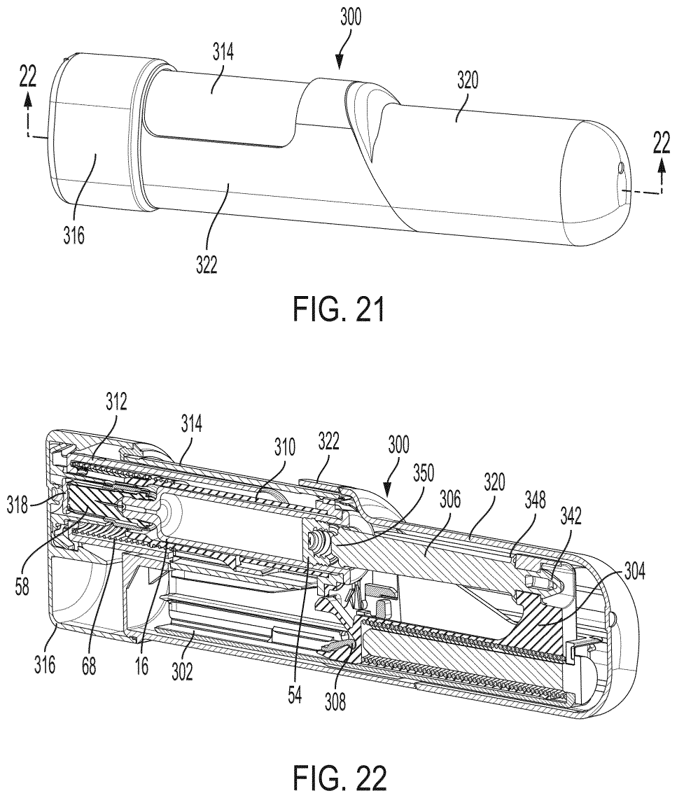

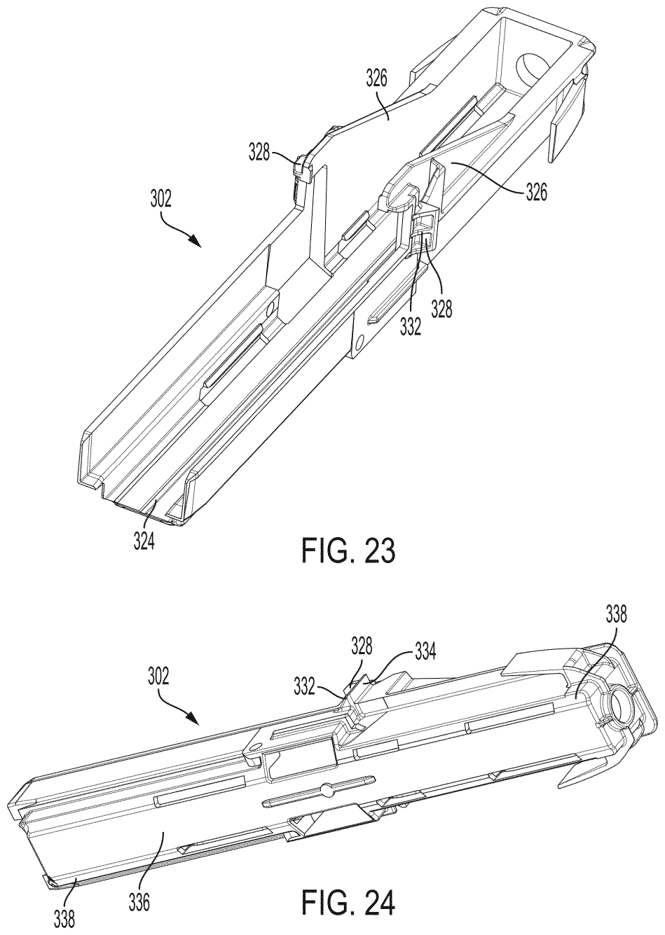

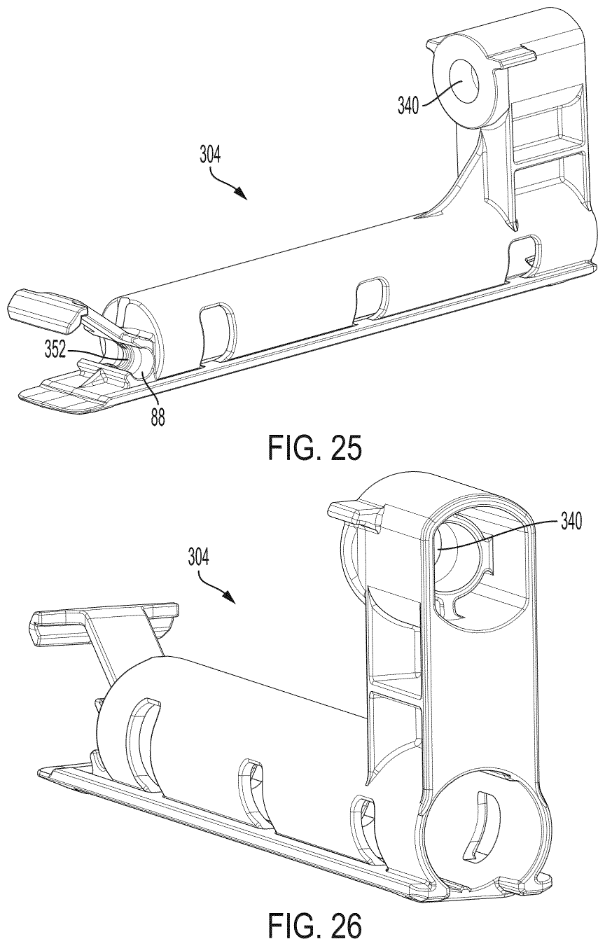

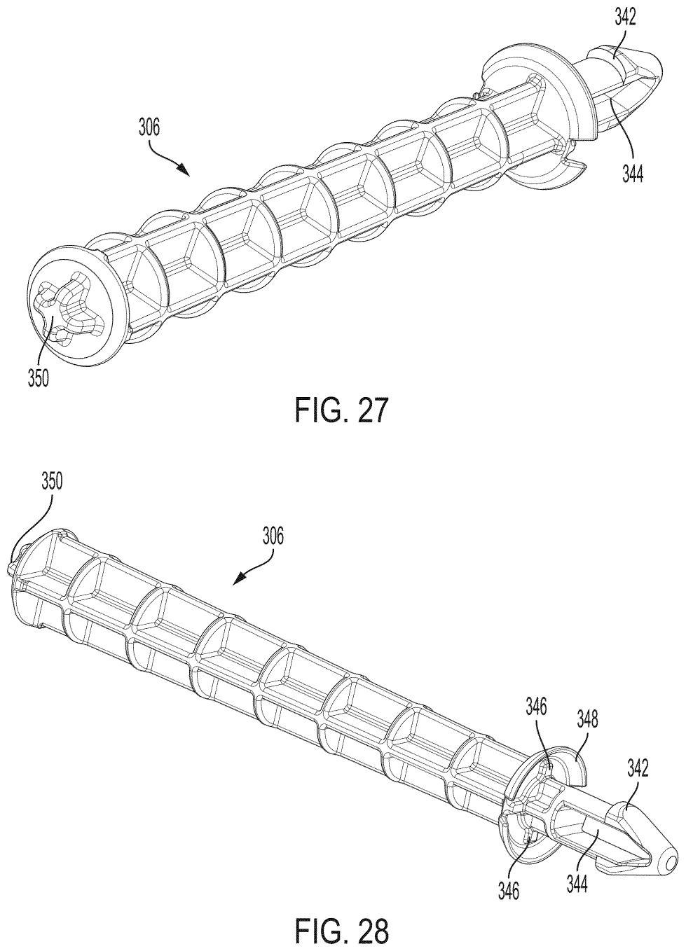

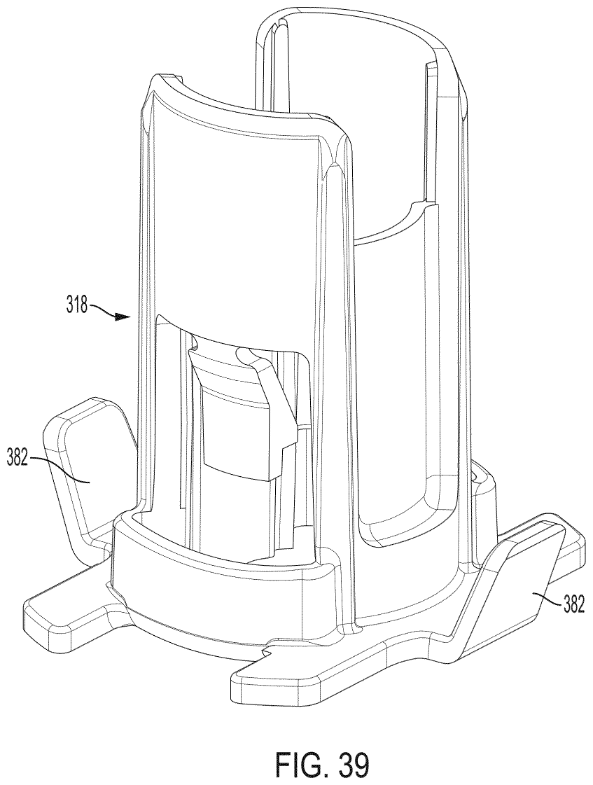

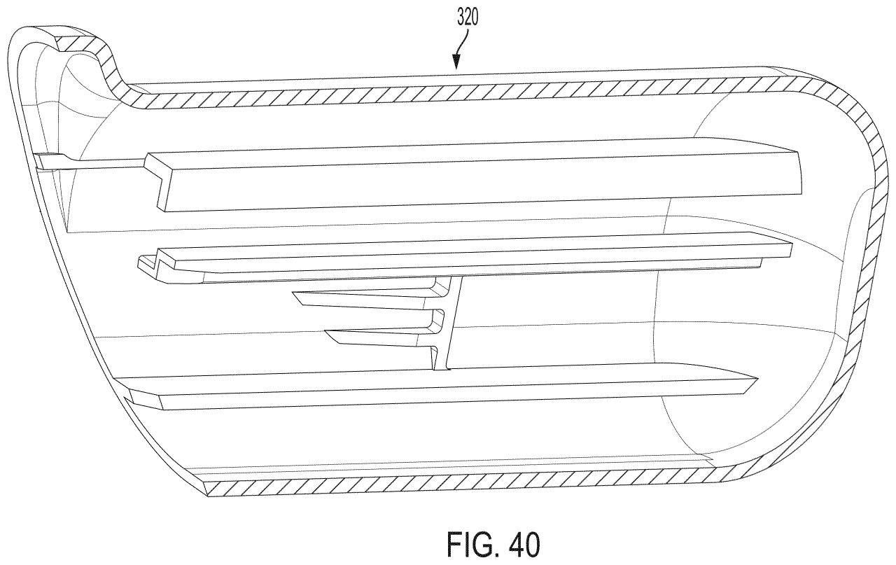

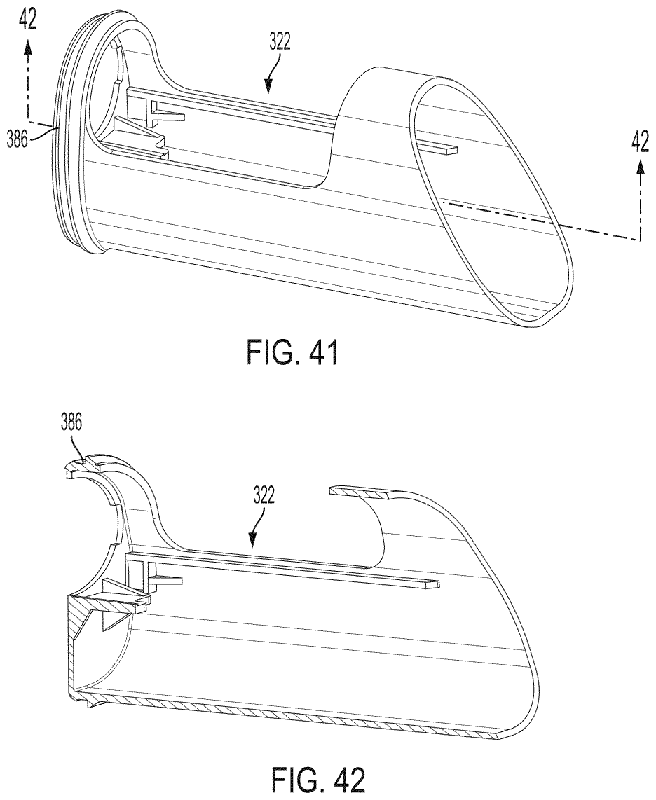

Referring to FIGS. 21-45, a drug delivery device 300 according to a further aspect of the present invention is shown. The drug delivery device 300 is similar to the drug delivery device 10 shown in FIGS. 1A-20, with certain differences discussed below in detail. The drug delivery device 300 includes, among other components, a motor body 302, a plunger body 304, a plunger rod portion 306, a lever actuation member 308, a syringe holder 310, a needle cover 312, a cassette body 314, a cap 316, a retainer 318, an upper housing shell 320, and a lower housing shell 322.

Referring to FIGS. 21-24, the motor body 302 is similar and functions similarly to the motor body 42 of FIGS. 1A-20, but further includes a longitudinal groove 324, reinforcing rib(s) 326, and cassette clip(s) 328. The longitudinal groove 324 is configured to receive a molding split line of the plunger body 304 to ensure smooth sliding between the motor body 302 and the plunger body 304. The reinforcing rib(s) 326 provide additional support for the pair of arms 260 of the motor body 302. The cassette clip(s) 328 is received by an opening(s) 330 defined by the cassette body 314 to secure the motor body 302 to the cassette body 314, which is discussed in more detail below. The cassette clip(s) 328 include an angled face 332 and a planar face 334, which is configured to allow insertion of the cassette clip(s) 328 into the opening(s) 330 of the cassette body 314, but prevent the easy removal of the cassette clip(s) 328 once inserted into the opening(s) 330 of the cassette body 314. A bottom surface 336 of the motor body 302 includes chamfered portions 338 to aid assembly of the device 300.

Referring to FIGS. 22 and 25-28, the plunger body 304 is formed separately from the plunger rod portion 306 rather than being formed integrally. Further, the device 300 does not include the plunger rod cover 92. The plunger body 304 defines an opening 340 that receives a plunger rod clip 342 of the plunger rod portion 306. The plunger rod clip 342 is barb-shaped and configured to be inserted into the opening 340 of the plunger body 304, but not easily removed from the opening 340, although other suitable shapes and configurations may be utilized. The plunger rod clip 342 defines a central opening 344, which allows the plunger rod clip 342 to compress as the plunger rod clip 342 is inserted into the opening 340 of the plunger body 304 and expand to its original shape once received within the plunger body 304. The plunger rod portion 306 includes a plunger body stop(s) 346 and a biasing member 348. The plunger body stop(s) 346, which may be one or more projections, contact the plunger body 304 when the plunger rod clip 342 is inserted into the opening 340 of the plunger body 304. The biasing member 348 engages the plunger body 304 during insertion of the plunger rod clip 342 into the opening 340 of the plunger body 304 and biases the plunger rod portion 306 toward the plunger body 304. The biasing member 348 provides additional leeway for insertion of the plunger rod clip 342 into the opening 340 of the plunger body 304 while ensuring there is no gap between the plunger body 304 and the plunger rod portion 306 after assembly. The biasing member 348 of the plunger rod portion 306 is annular, although other suitable shapes and configurations may be utilized.

The plunger rod portion 306 further includes a stopper interface 350 that is received by the stopper 54. The stopper interface 350 is a cruciform projection, although other suitable shapes and configurations may be utilized. The plunger rod portion 306 has a conical external shape configured to reduce stress on the syringe assembly 16, although other suitable shapes may be utilized. The plunger body 304 includes a lever rib 352 extending into the lever opening 88 of the plunger body 304. The lever rib 352 is configured to be received by the lever actuation member 308, as discussed in more detail below.

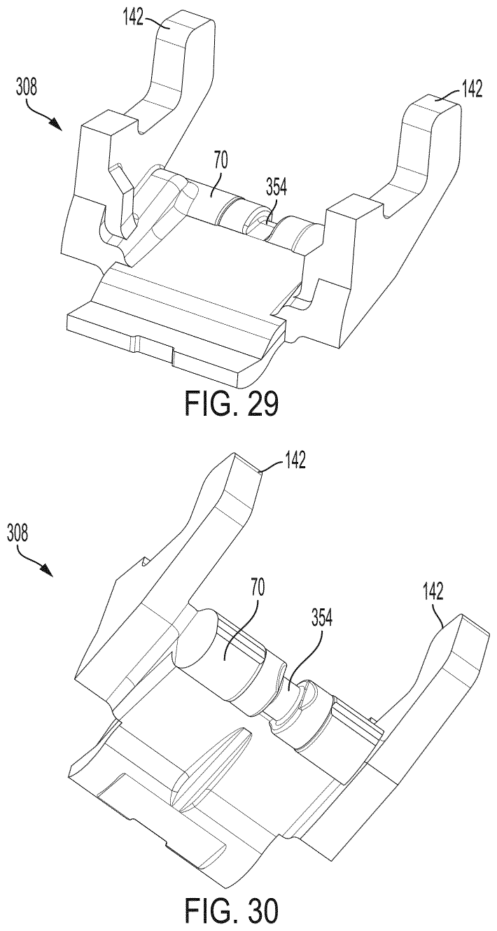

Referring to FIGS. 29 and 30, the lever actuation member 308 is similar to and functions similarly to the lever actuation member 44 described above and shown in FIGS. 1A-20. The lever actuation member 308, however, defines a groove 354 at the rotation axis 70 that receives the lever rib 352 of the plunger body 304. The engagement between the groove 354 and the lever rib 352 prevents relative lateral movement between the plunger body 304 and the lever actuation member 308. The needle cover contact surface 142 of the lever actuation member 308 includes a larger surface compared to the needle cover contact surface 142 of the lever actuation member 44 of FIGS. 1A-20.

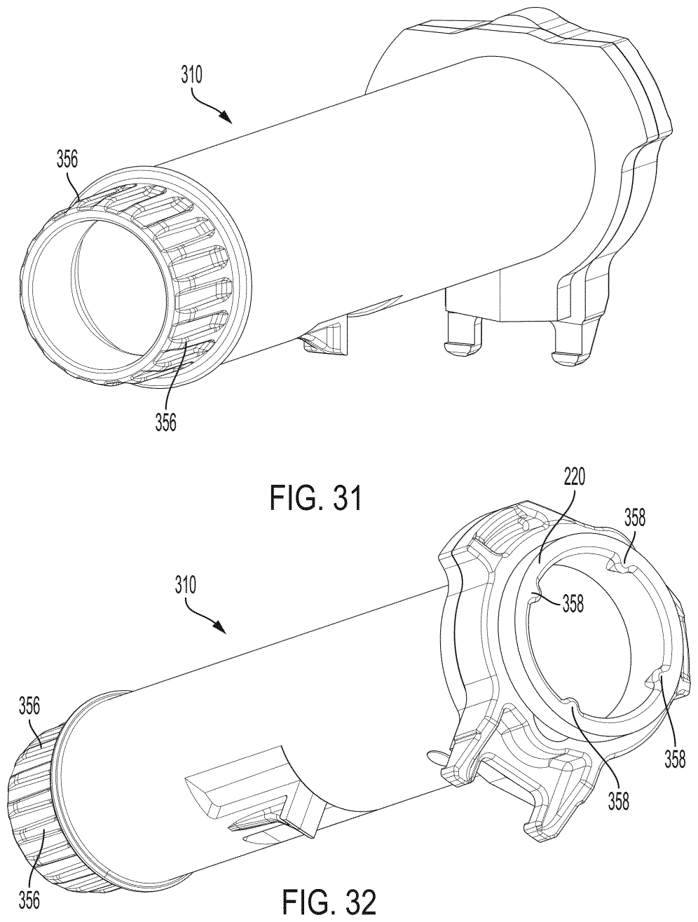

Referring to FIGS. 31 and 32, the syringe holder 310 is similar to and functions similarly to the syringe holder 24 of FIGS. 1A-20. The syringe holder 310, however, further includes a plurality of ribs 356 extending circumferentially around the syringe holder 310. The plurality of ribs engage the spring 68. The securing ring 220 of the syringe holder 310 further includes a plurality of projections 358 that extend radially inward. The plurality of projections 358 engage the syringe assembly 16 to remove any gap between the outer surface of the syringe assembly 16 and the syringe holder 310.The plurality of projections 358 are elastomeric and may compress when the syringe assembly 16 is received within the syringe holder 310.

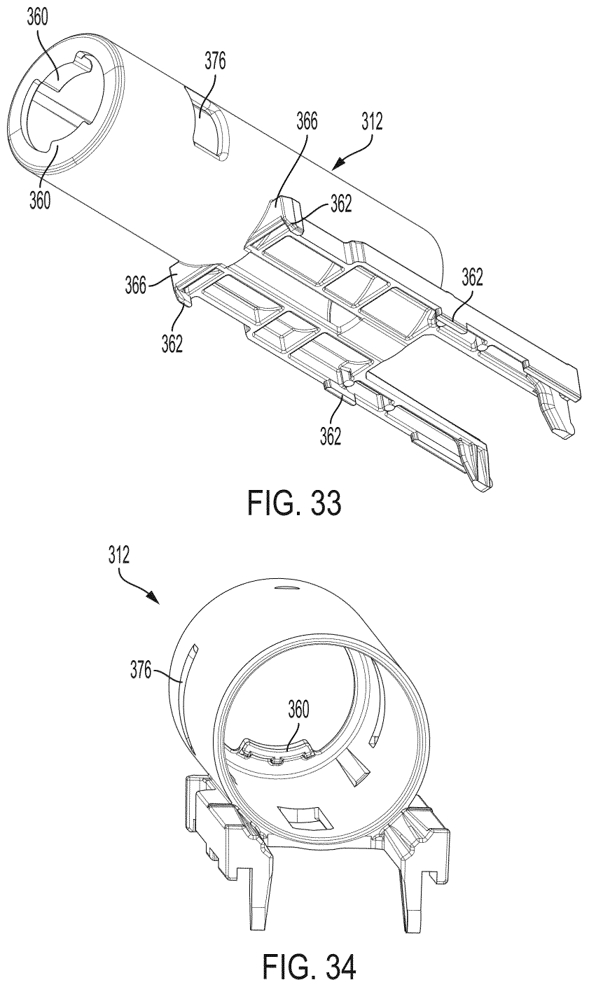

Referring to FIGS. 33 and 34, the needle cover 312 is similar to and functions similarly to the needle cover 22 of FIGS. 1A-20. The needle cover 312 includes a spring rib 360 which engages the spring 68 to hold the spring 68 between the needle cover 312 and the syringe holder 310. The needle cover 312 also includes a cassette rib(s) 362 to guide movement of the needle cover 312 relative to the cassette body 314.

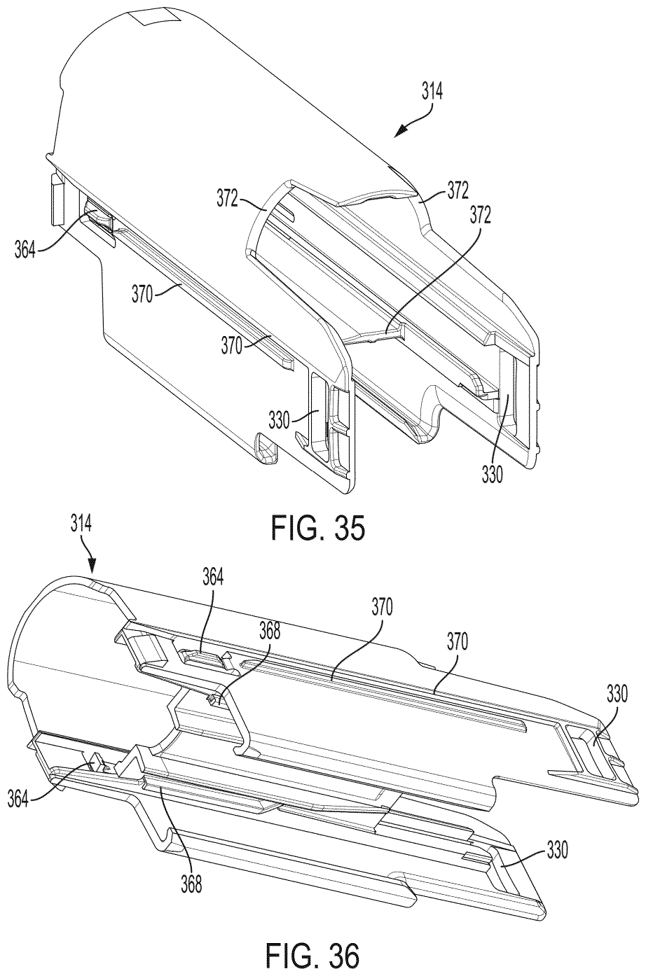

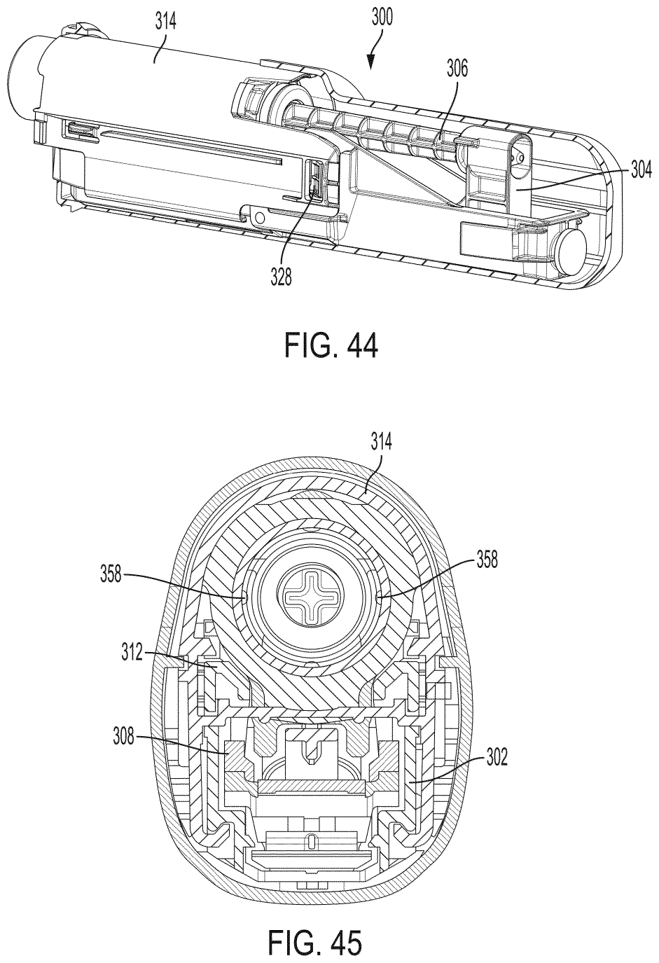

Referring to FIGS. 35, 36, 44, and 45, the cassette body 314 is similar to and functions similarly to the cassette body 26 of FIGS. 1A-20. As discussed above, the cassette body 314 includes the opening(s) 330 that receive the cassette clip(s) 328 of the motor body 302. The cassette body 314 includes a needle cover clip(s) 364 that engage clip surface(s) 366 of the needle cover 312. The clip surface(s) 366 of the needle cover 312 are planar, although other suitable shapes and configurations may be utilized. The needle cover clip(s) 364 are configured to restrict the axial movement of the needle cover 312 relative to the cassette body 314. The cassette body 314 further includes motor body rib(s) 368 and upper housing shell rib(s) 370, which are configured to engage corresponding portions of the motor body 302 and the upper housing shell 320 to aid in the assembly of the device 300. The cassette body 314 also includes syringe holder stop(s) 372, which are configured to engage portions of the syringe holder 310 to limit the axial movement of the syringe holder 310 relative to the cassette body 314. Although not shown in FIG. 44, the locking clip 64 may also be utilized with the drug delivery device 300.

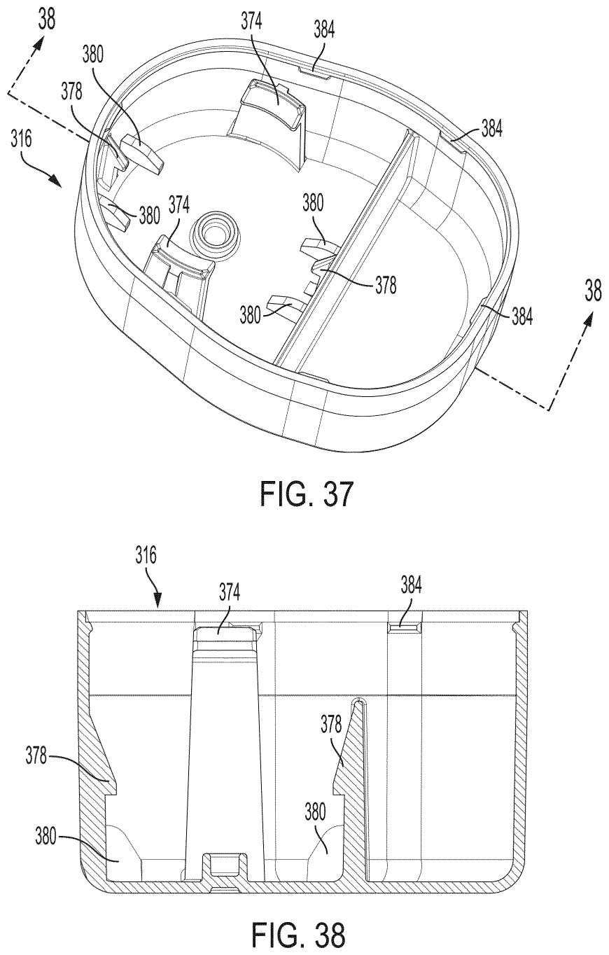

Referring to FIGS. 37-42, the cap 316 is similar to and functions similarly to the cap 18 described above and shown in FIGS. 1A-20. The cap 316 includes a protrusion(s) 374 that is received by a cap opening(s) 376 defined by the needle cover 312, which is positioned 90 degrees relative to the position of those elements of the cap 18 of FIGS. 1A-20. The protrusion(s) 374 of the cap 316 is configured to engage the needle cover 312 upon movement of the needle cover 312 from the pre-use position to the actuation position. For instance, with the device 300 in the storage position with the cap 316 secured to the lower housing shell 322, if the device is dropped or impacted to apply a force to the needle cover 312, the lever actuation member 308, and/or other component, the protrusion(s) 374 restricts movement of the needle cover 312, which prevents any unintended actuation of the device 300. The cap 316 further includes a retainer clip(s) 378 and a rib(s) 380 for engaging a wing(s) 382 of the retainer 318. The retainer clip(s) 378 and the rib(s) 380 secure the retainer 318 to the cap 316 and prevent any movement or wobbling of the retainer 318 relative to the cap 316. The retainer 318 is configured to remove the RNS 58 when the cap 316 is removed from the lower housing shell 322. The cap 316 includes a lower housing shell clip(s) 384 for engaging the lower housing shell 322 to secure the cap 316 to the lower housing shell 322. The upper housing shell 320 and the lower housing shell 322 are similar and function similarly to the upper housing shell 46 and the lower housing shell 28 discussed above and shown in FIGS. 1A-20. The lower housing shell 322, however, has a cap interface 386 to receive the lower housing shell clip(s) 384 of the cap 316.

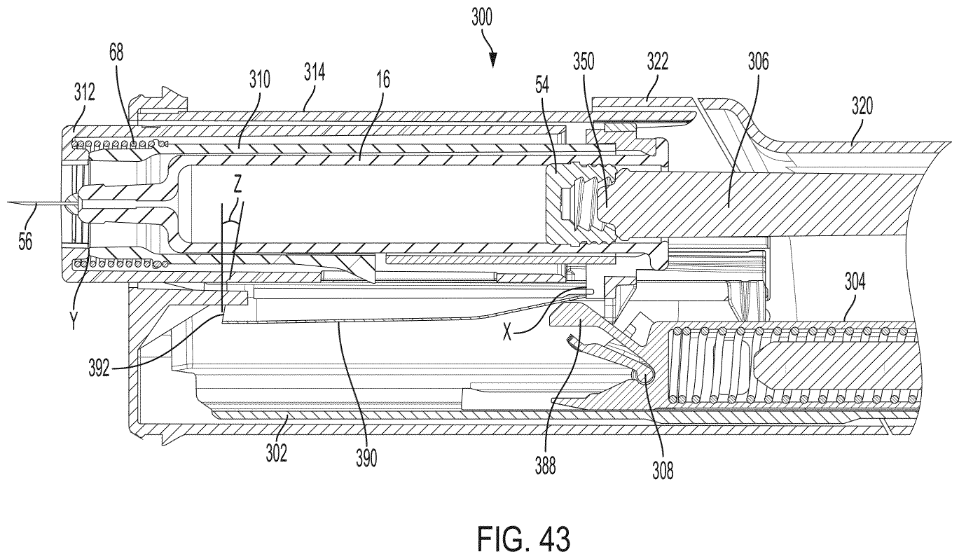

Referring to FIG. 43, the drug delivery device 300 is shown in an injection position. The injection depth of the cannula 56 is determined by contact between the syringe holder 310 and the cassette body 314 at point X and contact between the needle cover 312 and the syringe holder 310 at point Y.

Referring to FIG. 43, the drug delivery device 300 includes an audio indicator member 388, which is similar to and functions similarly to the audio indicator member 94 described above and shown in FIGS. 1A-20. In the same manner as the audio indicator member 94, which is described above, the audio indicator member 388 of the drug delivery device 300 is configured to provide an audible indication to a user when the device 300 transition to the post-use position. The audio indicator member 388 is configured to engage rib(s) 390 of the cassette body 314 when the device 300 is in the injection position thereby deflecting the audio indicator member 388. The audio indicator member 388 disengages from the rib(s) 390 of the cassette body 314 and contacts the lower housing shell 322 to provide an audible click when the drug delivery device 300 transition from the injection position to the post-use position. However, a distal end 392 of the rib(s) 390 of the cassette body 314 is angled rearward toward the upper housing shell 320, which beneficially provides a louder audible click compared to the arranged of the rib(s) 96 of the cassette body 26 discussed above in connection with FIGS. 1A-20.

In one aspect or embodiment, an angle Z of the distal end 392 of the rib(s) 390 of the cassette body 314 relative to a plane extending perpendicularly to a longitudinal axis of the device 300 is greater than 5 degrees. In one aspect or embodiment, the angle Z of the distal end 392 of the rib(s) 390 is greater than 10 degrees. In one aspect or embodiment, the angle Z of the distal end 392 is 25 degrees.

Elements of one disclosed aspect can be combined with elements of one or more other disclosed aspects to form different combinations, all of which are considered to be within the scope of the present invention.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A drug delivery device comprising:
a first subassembly comprising a cap, a needle cover, a syringe holder configured to receive a syringe assembly, a cassette body, and a lower housing shell, the cap receiving at least a portion of the needle cover;
a second subassembly comprising a drive assembly configured to move a stopper of a syringe assembly, and a motor body, the motor body receiving at least a portion of the drive assembly, the cassette body of the first subassembly is configured to receive at least a portion of the motor body; and
a locking clip configured to secure the first subassembly to the second subassembly upon assembly of the first subassembly to the second subassembly.

2. The drug delivery device of claim 1, further comprising a syringe assembly comprising a barrel, a stopper, a cannula, and a rigid needle shield receiving at least a portion of the cannula.

3. The drug delivery device of claim 1, wherein the locking clip comprises a body having a projection configured to engage a clip surface defined by the motor body of the second subassembly.

4. The drug delivery device of claim 1, wherein the cassette body defines a clip opening configured to receive a portion of the locking clip.

5. The drug delivery device of claim 1, wherein the locking clip is C-shaped.

6. The drug delivery device of claim 3, wherein the clip surface is defined by a recessed portion of the motor body, the clip opening of the cassette body is configured to be aligned with the clip surface of the motor body when the first subassembly is assembled with the second subassembly, a portion of the locking clip configured to extend through the clip opening of the cassette body and the recessed portion of the motor body with the projection of the locking clip engaging the clip surface of the motor body.

7. The drug delivery device of claim 1, wherein the cassette body defines first and second grooves and the motor body defines first and second ribs, the first groove of the cassette body configured to receive the first rib of the motor body, and the second groove of the cassette body configured to receive the second rib of the motor body.

8. The drug delivery device of claim 1, wherein the locking clip is formed integrally with the cassette body.

9. The drug delivery device of claim 8, wherein the locking clip comprises a first locking clip positioned on a first side of the cassette body and a second locking clip positioned on a second side of the cassette body.

10. The drug delivery device of claim 8, wherein the motor body defines a clip surface configured to engage the locking clip(s) of the cassette body.

11. The drug delivery device of claim 1, further comprising an upper housing shell configured to be positioned adjacent to the lower housing shell.

12. The drug delivery device of claim 9, wherein the motor body includes a first extension defining a first channel and the cassette body defines a second extension defining a second channel, the first channel of the motor body configured to receive the second extension of the cassette body.

13. The drug delivery device of claim 12, wherein the second channel of the cassette body is configured to receive the first extension of the motor body.

14. A method of assembling a drug delivery device comprising:
moving a first subassembly into engagement with a second subassembly, the first subassembly comprising a cap, a needle cover, a syringe holder configured to receive a syringe assembly, a cassette body, and a lower housing shell, the second subassembly comprising a drive assembly configured to move a stopper of a syringe assembly, and a motor body;
securing the first subassembly to the second subassembly with a locking clip; and
moving an upper housing shell into engagement with the second subassembly.

15. The method of claim 14, further comprising:
positioning a syringe assembly in the syringe holder of the first subassembly, the syringe assembly comprising a barrel, a stopper, a cannula, and a rigid needle shield receiving at least a portion of the cannula.

16. The method of claim 14, wherein the locking clip comprises a body having a projection and the motor body defines a clip surface, and wherein the cassette body defines a clip opening configured to receive a portion of the locking clip, the method further comprising:
inserting a portion of the locking clip through the clip opening of the cassette body and engaging the clip surface of the motor body to secure the cassette body to the motor body.

17. The method of claim 14, wherein the locking clip is formed integrally with the cassette body, and wherein the motor body defines a clip surface, the method further comprising:
positioning the motor body within the cassette body until the locking clip of the cassette body engages the clip surface of the motor body thereby securing the first subassembly to the second subassembly.
